(19) United States
(12) Patent Application Publication
(10) Pub. No.: US 2023/0079488 A1
(43) Pub. Date: Mar. 16, 2023

(54) Medical Dilator

(71) Applicant: Boston Scientific Medical Device Limited, Galway (IE)

(72) Inventors: John Paul Urbanski, Toronto (CA); Gareth Davies, Toronto (CA)

(21) Appl. No.: 17/760,337

(22) PCT Filed: Feb. 8, 2021
(86) PCT No.: PCT/IB2021/051008

§ 371 Date: Aug. 8, 2022

Related U.S. Application Data
(60) Provisional application No. 62/972,810, filed on Feb. 11, 2020.

(51) Int. Cl. A61B 18/14; A61M 29/02

(52) U.S. Cl. CPC A61B 18/1487; A61M 29/02; A61B 2018/00595

(57) ABSTRACT

An apparatus includes an elongated medical dilator including a cautery device configured to selectively form, by cauterization, a tissue passage through a tissue portion of a living body. The elongated medical dilator also includes a dilation device configured to dilate the tissue passage once the cautery device selectively formed the tissue passage.

### TECHNICAL FIELD

This document relates to (and is not limited to) the technical field of medical dilators, and more specifically, this document relates to the technical field of medical dilators including a synergistic combination of a cautery device and a dilation device (and method therefor).

### BACKGROUND

Known (existing) medical dilators are configured to form a hole (a passageway) extending through the tissue of a patient.

### SUMMARY

It will be appreciated that there exists a need to mitigate (at least in part) at least one problem associated with the existing (known) medical dilators (also called the existing technology). After much study of, and experimentation with, existing medical dilators, an understanding (at least in part) of the problem and its solution have been identified (at least in part) and are articulated (at least in part) as follows:

Known medical dilators are configured to impart a mechanical force to the tissue of a patient; the mechanical force (applied or imparted to the tissue) is utilized for forming a pilot hole (a passageway) extending through the tissue. Once the pilot hole is initially formed, further application of the mechanical force (to the pilot hole) expands the diameter of the pilot hole. For the case where the mechanical force is applied relatively aggressively to the tissue and/or the initially-formed pilot hole, known medical dilators, from time to time, may impart (inflict) inadvertent (unwanted) physical damage to the adjacently positioned tissue and/or the pilot hole; this unwanted condition may lead to unintended collateral damage to the tissue and/or the pilot hole, which may require further medical intervention or attention leading to prolonged operating-room time and costs. It will be appreciated that the pilot hole may be formed by another device (such as, a needle or wire positioned inside the medical dilator), and that the other device is not necessarily a component of the medical dilator, but may be an accessory for the medical dilator. For some cases, it will be appreciated that the pilot hole may be formed by the medical dilator (a mechanical dilator may be utilized to create the pilot hole and this case may increase the risk to the patient with relatively lesser control options for the surgeon).

During transseptal applications (in which known medical dilators are utilized for tissue dilation and sheath placement over a medical guidewire), a physician may need to deploy (via a device exchange procedure) a specialized device to perform an initial transseptal puncture in the tissue of the patient. A medical device exchange is a medical process (procedure) for removing one medical device (deployed on a medical guidewire that remains positioned in the body of a patient), and inserting (deploying) another medical device over (via) the medical guidewire. Known medical dilators may require at least one or more device exchanges (such as, exchanges between medical guidewires, medical sheaths, and/or medical puncture devices, etc.). Every medical device exchange (and device repositioning thereof) may involve unwanted risk, such as potentially risky exposure, for instance, to additional x-ray radiation to patient and/or the physician, etc. Therefore, to reduce the frequency of occurrence of medical device exchanges, it may be advantageous to provide a medical dilator including a synergistic combination of a cautery device and a dilation device (in which, in accordance with a preferred embodiment, the medical dilator is configured for deployment with a medical guidewire). For instance, the cautery device may be utilized for forming an initial pilot hole through the tissue (once the cautery device is activated); the dilation device may be utilized to expand the diameter of the initial pilot hole. In this arrangement, the medical dilator, may avoid at least one medical device exchange and/or improve the procedural efficiency and safety associated with medical dilators.

It may be desirable to provide a medical dilator including a cautery device and a dilation device each positioned at a distal end portion of the medical dilator. Preferably, the medical dilator is configured to (A) accommodate a guidewire exchange via an inner channel defined by the medical dilator, and/or (B) accommodate sheath exchange via an outer surface of the elongated medical dilator. It may be advantageous to provide the medical dilator including a cautery device configured to perform a puncture (through the tissue) to improve procedural efficiency. It may be desirable to provide the medical dilator having a cautery device built-in, or integrated with, the medical dilator (thereby avoid the deployment of a cutting device that is separate from the medical dilator, such as via a process for device exchange).

To mitigate, at least in part, at least one problem associated with the existing technology, there is provided (in accordance with an aspect) an apparatus. The apparatus includes, and is not limited to, (comprises) an elongated medical dilator including a cautery device configured to selectively form, by cauterization, a tissue passage through a tissue portion of a living body. The elongated medical dilator also includes a dilation device positioned relative to the cautery device. The dilation device is configured to dilate (to further mechanically open) the tissue passage once the cautery device has selectively formed the tissue passage.

To mitigate, at least in part, at least one problem associated with the existing technology, there is provided (in accordance with an aspect) an apparatus. The apparatus includes, and is not limited to, an elongated medical dilator including a cautery device configured to selectively form, by cauterization, a tissue passage through a tissue portion of a living body once the elongated medical dilator is received into a confined space defined by the living body, and the cautery device is positioned proximate to the tissue portion. The elongated medical dilator includes a dilation device positioned relative to the cautery device. The dilation device is also configured to dilate the tissue passage once the cautery device selectively formed the tissue passage, and once the dilation device is urged to move toward to make intimate (and forced) contact with the tissue passage formed by the cautery device.

To mitigate, at least in part, at least one problem associated with the existing technology, there is provided (in accordance with an aspect) a method. The method includes, and is not limited to, (comprises): (A) utilizing a cautery device of an elongated medical dilator for selectively forming, by cauterization, a tissue passage through a tissue portion of a living body, and (B) utilizing a dilation device of the elongated medical dilator, in which the dilation device is positioned relative to the cautery device, for dilating the tissue passage once the cautery device selectively formed the tissue passage.

To mitigate, at least in part, at least one problem associated with the existing technology, there is provided (in accordance with an aspect) a method. The method includes, and is not limited to, (comprises) (A) utilizing a cautery device of an elongated medical dilator for selectively forming, by cauterization, a tissue passage through a tissue portion of a living body once the elongated medical dilator is received into a confined space defined by the living body, and the cautery device is positioned proximate to the tissue portion; and (B) utilizing a dilation device of the elongated medical dilator, in which the dilation device is positioned relative to the cautery device, for dilating the tissue passage once the cautery device selectively formed the tissue passage, and the dilation device is urged to move toward, and to make intimate contact with, the tissue passage formed by the cautery device.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1A, FIG. 1B and FIG. 1C depict a front view (FIG. 1A), a side perspective view (FIG. 1B), and a cross-sectional view (FIG. 1C) taken along a cross-sectional line A-A of FIG. 1A of embodiments of an elongated medical dilator; and

FIG. 2A, FIG. 2B and FIG. 2C depict front views (FIG. 2A and FIG. 2B) of embodiments of the elongated medical dilator of FIG. 1A, and a side cross-sectional view (FIG. 2C) taken along a cross-sectional line B-B of FIG. 2A; and

FIG. 2D, FIG. 2E and FIG. 2F depict front views (FIG. 2D and FIG. 2E) of embodiments of the elongated medical dilator of FIG. 1A, and a side cross-sectional view (FIG. 2F) taken along a cross-sectional line C-C of FIG. 2D; and

FIG. 2G, FIG. 2H and FIG. 2I depict front views (FIG. 2G and FIG. 2H) of embodiments of the elongated medical dilator of FIG. 1A, and a side cross-sectional view (FIG. 2I) taken along a cross-sectional line D-D of FIG. 2G; and

FIG. 3A, FIG. 3B and FIG. 3C depict a cross-sectional view (FIG. 3A) of an embodiment of the elongated medical dilator of FIG. 1, a side view (FIG. 3B) and a perspective view (FIG. 3C) of the embodiments of the elongated medical dilator of FIG. 3A; and

FIG. 3D, FIG. 3E, FIG. 3F and FIG. 3G depict a cross-sectional view (FIG. 3D) of an embodiment of the elongated medical dilator of FIG. 1, a side view (FIG. 3E), a front view (FIG. 3F) and a side perspective view (FIG. 3G) of the embodiments of the elongated medical dilator of FIG. 3D; and

FIG. 4A, FIG. 4B, FIG. 4C and FIG. 4D depict a work flow using the embodiment of the elongated medical dilator of FIG. 1A; and

FIG. 5A, FIG. 5B and FIG. 5C depict cross-sectional views of the embodiments of the elongated medical dilator of FIG. 1A.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

| | |
|---|---|
| outer surface | 101 |
| medical dilator | 102 |
| longitudinal axis | 103 |
| longitudinal axis extending | 103 |
| cautery device | 104 |
| thermal energy | 105 |
| dilation device | 106 |
| face portion | 107 |
| dilator length | 108 |
| electrically-conductive material | 109 |
| dilator cavity | 110 |
| electrical conductor | 111 |
| Cautery device hole | 113 |
| Cautery device body | 115 |
| flexible section | 117 |
| section | 119 |
| medical sheath | 200 |
| tissue passage | 900 |
| tissue portion | 902 |
| heart | 903 |
| living body | 904 |
| safety gap | 905 |
| space | 906 |
| outer gap | 907 |
| medical guidewire assembly | 908 |
| inner gap | 909 |
| movement direction | 911 |

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

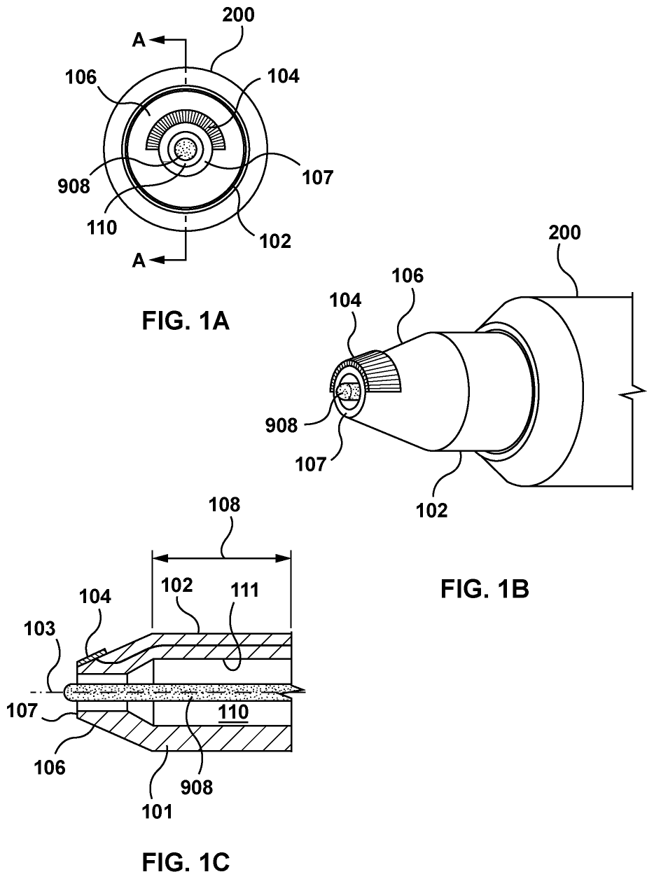

FIG. 1A, FIG. 1B and FIG. 1C depict a front view (FIG. 1A), a side perspective view (FIG. 1B), and a cross-sectional view (FIG. 1C) taken along a cross-sectional line A-A of FIG. 1A of embodiments of an elongated medical dilator 102.

Referring to the embodiments as depicted in FIG. 1A and FIG. 1B, an apparatus includes and is not limited to (comprises) an elongated medical dilator 102. The elongated medical dilator 102 may include (and is not limited to) a transseptal dilator configured for utilization with a medical procedure related to the heart of a patient, etc., and/or any equivalent thereof. The elongated medical dilator 102 is biocompatible, maneuverable, and robust. The elongated medical dilator 102 includes (preferably) a synergistic combination of a cautery device 104 and a dilation device 106. The cautery device 104 is configured to selectively form, by cauterization, a tissue passage 900 through a tissue portion 902 of a living body 904 (once the cautery device 104 is positioned proximate to the tissue portion 902 and once the cautery device 104 is actuated). The definition of cauterization includes utilization of a medical tool (such as the cautery device 104) for the management (application and/or removal) of thermal energy proximate to living tissue for the purpose of forming a passageway through the living tissue while sealing off blood vessels in the living tissue and preventing unwanted bleeding from the living tissue (thereby promoting easier healing). Cauterization may include vaporization of tissue (by removal of moisture from tissue). The cautery device 104 may utilize a relatively lower electrical current level or a relatively higher electrical current level.

Referring to the embodiments as depicted in FIG. 1A and FIG. 1B, the cautery device 104 is positioned relative to the dilation device 106. For instance, the cautery device may be positioned adjacent to, or positioned over the dilation device, etc. The dilation device 106 is configured to dilate (to mechanically expand or ream) the tissue passage 900 once the cautery device 104 selectively forms the tissue passage 900 (that is, after the cautery device 104 has selectively formed the tissue passage 900, then the dilation device 106 is moved toward, to physically and intimately contact, the tissue passage 900 so as to further enlarge the size of the tissue passage 900). For instance, the dilation device 106 may include a dilation tip, a distal tip portion and/or any equivalent thereof. At least one technical effect of the elongated medical dilator 102 includes a reduction of time for performing a medical procedure, a reduction in medical operating steps to be executed by a medical doctor and support staff. The elongated medical dilator 102 provides a single apparatus configured to (A) form the tissue passage 900 and (B) dilate the size of the tissue passage 900 (once the tissue passage 900 is formed); utilization of the elongated medical dilator 102 avoids deployment of separate medical devices: that is, one medical device for forming the tissue passage 900, and another medical device for dilating the size of the tissue passage 900 (once the tissue passage 900 is formed). The deployment of two separate medical devices increases surgical time, increases the potential for medical complications during surgery, and/or increases cost (more time in a surgical room leads to increased hospital overhead and/or operating costs). The elongated medical dilator 102 disclosed herein results in simplification of medical procedures and/or costs.

Referring to the embodiments as depicted in FIG. 1A and FIG. 1B, the elongated medical dilator 102 has a measure (degree) of stiffness (hardness). The body of the elongated medical dilator 102 may have sufficient stiffness so that the elongated medical dilator 102 may be advanced (within the body of the patient) and torqued within the patient, with sufficient flexibility to be atraumatic (to the patient). The elongated medical dilator 102 may include a variety (blend) of various materials to modify the mechanical properties of the elongated medical dilator 102 (that is, its bending stiffness or bendability) along a length of the elongated medical dilator 102. For instance, several classes of bodies for the elongated medical dilator 102 may be considered. One class of body of the elongated medical dilator 102 includes a dilator constructed using one durometer of polymer extrusion from the proximal hub to distal tip of the elongated medical dilator 102. Another class includes a flexible instance of the elongated medical dilator 102 in which the stiffness of the elongated medical dilator 102 is selectively modified along its longitudinal length that allows the elongated medical dilator 102 to be deflected by an ancillary device, such as a steerable medical sheath (known and not depicted, such as disclosed in US Patent publication US 2016/0175009A1 (Inventors: John Paul URBANSKI, et. al.; Title: METHODS AND DEVICES FOR PUNCTURING TISSUE; Published: 23 Jun. 2016)). A specific example is the elongated medical dilator 102 with a High-Density Polyethylene (HDPE) proximal shaft and a Low-Density Polyethylene (LDPE) distal section (when used with a steerable sheath with a deflectable distal end, the tip of the elongated medical dilator 102 may be precisely positioned and set against the tissue of the patient). Another class of the elongated medical dilator 102 is configured to be shapeable or reinforced as disclosed in Patent publication WO 2018/083599 A1 (Inventors: John Paul URBANSKI, et al.; Title: METHODS AND DEVICES FOR PUNCTURING TISSUE; Published: 2018 May, 11). For instance, the elongated medical dilator 102 may be constructed using a combination of metal, such as SAE (Society Automotive Engineers) TYPE 304 stainless steel, and a polymer extrusion (such as, polyethylene). The metal shaft may also provide an electrical pathway to the cautery device 104.

Referring to the embodiments as depicted in FIG. 1A and FIG. 1B, the cautery device 104 is (preferably) configured to emit radio frequency energy from the proximal end of the elongated medical dilator 102 to the cautery device 104 mounted at the distal end of the dilation device 106. The energy transmitted or conveyed to the cautery device 104 should not affect devices or fluid outside or inside of the elongated medical dilator 102. In order to provide electrical safety to the patient and the user, and additionally to provide effective current delivery to the cautery device 104, a high voltage line through the elongated medical dilator 102 may require a significant amount of electrical insulation.

Referring to the embodiment as depicted in FIG. 1A and FIG. 1B, an electrical insulation material may be appropriate for the body of the elongated medical dilator 102. The electrical insulation may be fabricated with reflowed extrusions, multi-lumen extrusions with adequate wall thickness. Materials such as PTFE (Polytetrafluoroethylene), polyethylene, nylon, etc. may be useful in this regard. PTFE may be a preferred material for high voltage (electrical) insulation. PTFE is also available in a heat shrink format to ensure conformal adherence to wire mandrels with efficient use of available space (that is, to mitigate space consumed by voids between wires and hollow extrusion lumens). For instance, the elongated medical guidewire assembly 908 having an outer diameter of about 0.035 inches may require an effective insulation of about 0.003 inches of wall thickness of PTFE to satisfy current leakage requirements of electrosurgical medical standards (this may not take into account material or manufacturing variability, and as such, conductor cross-sectional area may be minimized and insulation deliberately oversized to ensure safety and performance). Additionally, it is desirable (though not necessary) for the external surface of the elongated medical dilator 102 to be smooth and lubricious to facilitate passage through vessels, tissue and other devices, etc. Additionally, it is desirable (though not necessary) for the external surface of the elongated medical guidewire assembly 908 to be smooth and lubricious to facilitate passage through vessels, tissue and other devices, etc.

Referring to the embodiments as depicted in FIG. 1A and FIG. 1B, the cautery device 104 may include a metallic alloy of stainless steel, nitinol, platinum and iridium blends, or a mix of the above, and any equivalent thereof. The cautery device 104 may be configured to operate under an electrocautery process. The cautery device 104 is configured to form (create) a pilot hole in the tissue, and then the dilation device 106 is utilized (after an initial opening is made by the cautery device 104) for tearing propagation and dilation with mechanical force. Advantageously, by forming the pilot hole (by utilizing the cautery device 104), relatively less mechanical trauma may be inflicted to the tissue for forming the passageway through the tissue. The total exposed metallic/conductive area of the cautery device 104 may be from about 1.2 mm\hat{ }2 to about 2.4 mm\hat{ }2 (millimeters squared) to ensure high current density for the case where about 270 Vrms to about 400 Vrms (Volts root mean square) is delivered in a unipolar manner (that is, to a grounded patient) to achieve initial puncture (formation of the pilot hole) of the tissue. It will be appreciated that the term “cautery” may include electrocautery, electrosurgery, and any equivalent thereof. In addition, the term “cautery” may also include any suitable thermal-based process configured for facilitating a cautery process, and any equivalent thereof.

Referring to the embodiments as depicted in FIG. 1A and FIG. 1B, the cautery device 104 may be formed to provide a blunt surface so that the cautery device 104 does not mechanically puncture the tissue inadvertently, or skive ancillary devices (such as the linings of sheaths), and may become effectively sharp (activated) when energy is applied to the cautery device 104. The cautery device 104 may be pre-manufactured and then embedded into an insulating material (a portion, such as a distal end portion) of the elongated medical dilator 102. Alternatively, the entire distal section of the cautery device 104 may be conductive, and then selectively insulated by shielding with a polymer (such as PTFE). It is may be desirable for the insulating material surrounding the cautery device 104 to withstand the shear stresses of tissue dilation, while also withstanding the high temperatures of energy delivery.

Referring to the embodiment as depicted in FIG. 1A and FIG. 1B, it may be desirable for the thermal energy (or RF energy) delivery to the cautery device 104 to not adversely affect devices and/or fluid inside of the elongated dilator cavity 110 (the lumen) of the elongated medical dilator 102. To provide electrical safety to the patient and the user, and additionally to provide effective energy (electrical current) delivery to the cautery device 104, a high voltage electrical conductor (wire or line) through the body of the elongated medical dilator 102 may require a significant amount of electrical insulation. The electrical insulation may be important near the distal tip of the cautery device 104, where the distal inner diameter of the elongated medical dilator 102 may approach (closely fit to) the outer diameter of the elongated medical dilator 102. For example, the elongated medical dilator 102 may have an inner diameter of about 0.050 inches or greater in their proximal section, and the very tip of the elongated medical dilator 102 may be tapered down to an inner diameter of about 0.036 inches for smoother transitions over the elongated medical guidewire assembly 908 having about a 0.035 inch outer diameter. About 0.001 inch to about 0.002 inch diametrical clearance is possible for compatibility with a certain size of the elongated medical guidewire assembly 908.

Referring to the embodiment as depicted in FIG. 1A and FIG. 1B, an apparatus includes and is not limited to (comprises) an elongated medical dilator 102. The elongated medical dilator 102 includes a synergistic combination of a cautery device 104 and a dilation device 106. The cautery device 104 is configured to selectively form, by cauterization, a tissue passage 900 through a tissue portion 902 of a living body 904 (once the elongated medical dilator 102 is received into a confined space 906 defined by the living body 904, and the cautery device 104 is positioned proximate to the tissue portion 902). The dilation device 106 is positioned relative to the cautery device 104. The dilation device 106 is configured to dilate the tissue passage 900 (once the cautery device 104 selectively formed the tissue passage 900, then the dilation device 106 is urged to move toward, and make intimate contact with, the tissue passage 900 formed by the cautery device 104 for the purpose of mechanically enlarging the tissue passage 900).

Referring to the embodiment as depicted in FIG. 1C, the elongated medical dilator 102 defines an elongated dilator cavity 110 (lumen) extending along a length of the elongated medical dilator 102. The elongated dilator cavity 110 is configured to receive an elongated medical guidewire assembly 908 therein, in which the elongated medical guidewire assembly 908 is configured to be inserted into the confined space 906 defined by the living body 904.

Referring to the embodiment as depicted in FIG. 1C, the elongated medical dilator 102 includes a distal tip having a leading face portion 107. The cautery device 104 is fixedly positioned at the leading face portion 107 of the distal tip of the elongated medical dilator 102.

Referring to the embodiment as depicted in FIG. 1C, the cautery device 104 includes an electrode.

Referring to the embodiment as depicted in FIG. 1C, the elongated electrical conductor 111 (electrical wire) is aligned along a length of the elongated medical dilator 102.

Referring to the embodiment as depicted in FIG. 1C, the elongated electrical conductor 111 (electrical wire) is aligned along a length of a body portion of the elongated medical dilator 102.

Referring to the embodiment as depicted in FIG. 1C, the elongated medical dilator 102 includes a proximal terminal. The cautery device 104 is configured to receive radio frequency energy (RF energy) from the proximal terminal via an elongated electrical conductor 111 (once the proximal terminal is selectively connected to a radio frequency energy source (an RF energy source), and the proximal terminal receives the radio frequency energy from the radio frequency energy source).

Referring to the embodiment as depicted in FIG. 1C, the elongated medical dilator 102 is configured to be received into a confined space 906 defined by a living body 904.

The elongated medical dilator 102 including a cautery device 104 configured to be positioned proximate to a tissue portion 902 of the living body 904 once the elongated medical dilator 102 is received into the confined space 906 defined by the living body 904. The cautery device 104 is also configured to selectively cauterize the tissue portion 902 of the living body 904; this is done in such a way that the cautery device 104, once actuated, forms, by cauterization, a tissue passage 900 through the tissue portion 902 once the cautery device 104 is positioned proximate to the tissue passage 900 and is selectively actuated. The elongated medical dilator 102 also includes a dilation device 106 (a dilation tip, a distal tip portion) positioned relative to the cautery device 104. The dilation device 106 is configured to be received into the confined space 906 defined by the living body 904. The dilation device 106 is also configured to support the cautery device 104. The dilation device 106 is configured to dilate (to expand) the tissue passage 900 once the cautery device 104 selectively formed the tissue passage 900; then once the tissue passage 900 is formed by the cautery device 104, the dilation device 106 is urged to move toward, and to make intimate and aggressive contact with, the tissue passage 900 formed by the cautery device 104 (for the purpose of enlarging the size of the tissue passage 900).

Referring to the embodiment as depicted in FIG. 1C, the elongated medical dilator 102 has a distal end portion and a dilation device 106 extending from the distal end portion. The elongated medical dilator 102 defines an elongated dilator cavity 110 (extending between the distal end portion and a proximal tip, from end to end of the elongated medical dilator 102). The elongated dilator cavity 110 is configured to receive the elongated medical guidewire assembly 908 configured to be inserted into a confined space 906 defined by a living body 904. The elongated medical dilator 102 also has a proximal tip that is spaced apart from the distal end portion. The elongated medical dilator 102 also has a proximal terminal positioned at the proximal tip, and the proximal terminal is configured to be selectively connected to an energy source (known and not depicted). The proximal terminal is configured to receive energy from the energy source once the proximal terminal is selectively connected to the energy source. The cautery device 104 is placed at a leading face portion 107 (leading edge) of the distal tip of the elongated medical dilator 102. The cautery device 104 is electrically coupled, via an elongated electrical conductor 111 (conductor) aligned along a length of the elongated medical dilator 102, to the proximal terminal. The cautery device 104 is configured to receive the energy from the proximal terminal once the proximal terminal is selectively connected to the energy source, and the proximal terminal receives the energy from the energy source. The cautery device 104 is configured to puncture through adjacently positioned tissue of the patient once the distal electrode receives the energy from the energy source.

Referring to the embodiments as depicted in FIG. 1A and FIG. 1B, the elongated medical dilator 102 includes the cautery device 104 and the dilation device 106. The dilation device 106 and the cautery device 104 are positioned at a distal end portion of the elongated medical dilator 102. The dilation device 106 and the cautery device 104 are positioned proximate to each other. Preferably, the cautery device 104 is positioned over at least a portion of the dilation device 106. The elongated medical dilator 102 is configured to deliver (emanate) thermal energy (or radio frequency energy) from a distal tip of the elongated medical dilator 102. The cautery device 104 (such as, an electrode) is placed at a leading face portion 107 (leading edge) of the elongated medical dilator 102. The cautery device 104 is configured to puncture through tissue (so that the doctor may avoid removal of the medical dilator 102 and subsequent deployment of a separate puncture device, such as a needle, etc.).

Referring to the embodiment as depicted in FIG. 1B, the cautery device 104 is configured to ablate. The cautery device 104 is configured to (shaped to) avoid formation of a tissue core (tissue coring) from the tissue (to be cauterized) once the cautery device 104 is activated (to avoid the formation of a stray tissue portion that might travel in the bloodstream). It will be appreciated that it may be desirable to avoid embolic stroke. An embolic stroke refers to the blockage of an artery by an embolus, a traveling particle or debris in the arterial bloodstream originating from elsewhere. Tissue coring may occur when applying electrocautery through an open-ended electrode (round or circular-shaped electrode).

Referring to the embodiments as depicted in FIG. 1B, a medical sheath 200 is an enveloping structure (a tubular instrument) through which a medical device (special obturators or cutting instruments, etc.) can be passed (such as the elongated medical dilator 102), etc. The medical sheath 200 may include a tube configured to be placed in an artery or vein during a procedure to help a doctor with insertion of catheter, etc. The medical sheath 200 defines an elongated interior passageway configured to receive the elongated medical dilator 102. The elongated medical dilator 102 may be utilized in the following way: during transseptal puncture applications, or applications that involve catheterization of body cavities where a sheath must be positioned using a guidewire and dilator in the body through a thin, membranous tissue. The medical sheath 200 defines an elongated interior passageway configured to receive the elongated medical dilator 102; the geometry of the entrance of the medical sheath 200 is adapted to avoid unwanted electrical contact between the elongated medical guidewire assembly 908 and the cautery device 104.

Referring to the embodiments as depicted in FIG. 1C, the cautery device 104 is positioned on the distal end of the elongated medical dilator 102. The geometry of the cautery device 104 is configured to not form a core tissue during activation of the cautery device 104 (such as RF puncture); for instance, the electrodes of the cautery device 104 do not circumscribe an area or volume of the tissue. The body of the elongated medical dilator 102, excluding the cautery device 104, is electrically insulating to the external surfaces (such as tissue, blood ancillary sheaths, and/or the physician). Further, the body of the elongated medical dilator 102 is electrically insulating to the internal lumen and devices therein (such as needles, wires, etc.). An insulated electrical connection is positioned between the cautery device 104 and a proximal hub of the elongated medical dilator 102. The elongated medical dilator 102 includes a proximal hub connection configured for selective connection to a generator or recording system (known and not depicted). A working length and diameter of the elongated medical dilator 102 is sufficient for sheath/device exchange (if so desired). The outer diameter of the elongated medical guidewire assembly 908 may have an outer diameter from about 0.014 inches to about 0.038 inches, preferably an outer diameter from about 0.032 inches to about 0.035 inches, etc. The outer diameter of the elongated medical dilator 102 may range from about 4 Fr to about 30 Fr, or from about 8 Fr to about 12 Fr (French catheter scale sizing), etc.

Referring to the embodiments as depicted in FIG. 1C, the dilation device 106 is configured to mechanically ream a passageway extending through the tissue (of the patient). The dilation device 106 may include a sloped surface extending from the distal portion of the elongated medical dilator 102. Preferably, the sloped surface of the dilation device 106 is aligned at an obtuse angle relative to the outer surface 101 of the elongated medical dilator 102. The sloped surface of the dilation device 106 extends from the outer surface of the elongated medical dilator 102 toward a longitudinal axis 103 extending through the elongated medical dilator 102.

Referring to the embodiments as depicted in FIG. 1C, the elongated medical dilator 102 defines an elongated dilator cavity 110 (lumen) (extending along a dilator length 108 of the elongated medical dilator 102 and/or extending along the longitudinal axis 103 of the elongated medical dilator 102). The elongated dilator cavity 110 is sized (configured to receive) the elongated medical guidewire assembly 908. The elongated medical guidewire assembly 908 may include, for instance, a plain wire, etc. The elongated medical dilator 102 and the elongated medical guidewire assembly 908 are movable relative to each other. For the case where the elongated medical guidewire assembly 908 is held in a fixed position (relative to the tissue of the patient), the elongated medical dilator 102 is movable. For the case where the elongated medical dilator 102 is held in a fixed position (relative to the tissue of the patient), the elongated medical guidewire assembly 908 is movable (retractable). For instance, once the tissue passage is formed by the elongated medical dilator 102, the elongated medical guidewire assembly 908 may be moved into and past the tissue passageway (and the elongated medical dilator 102 may be removed or retracted, etc.).

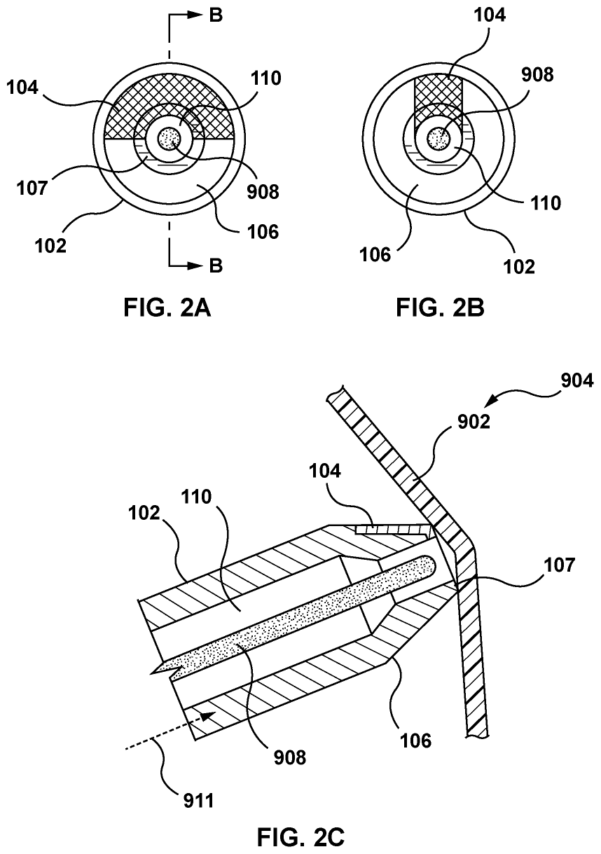

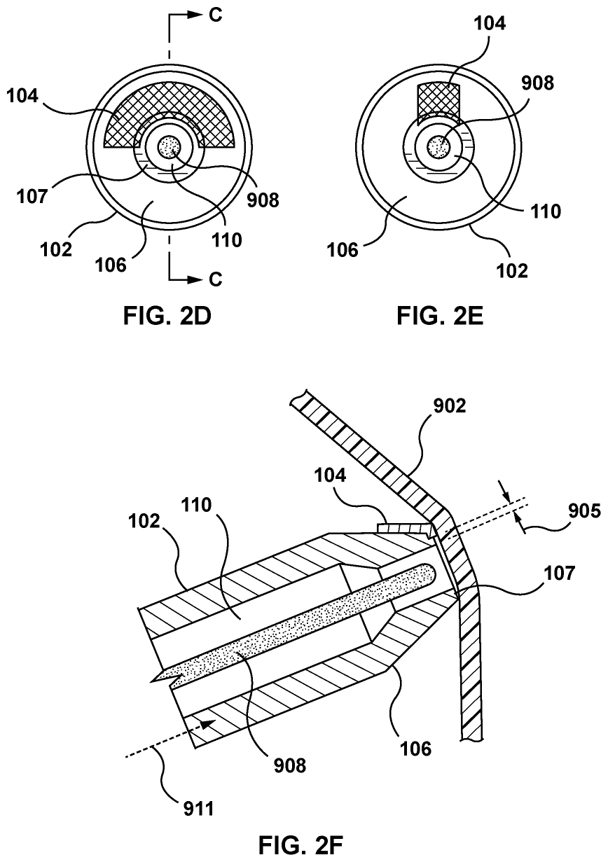

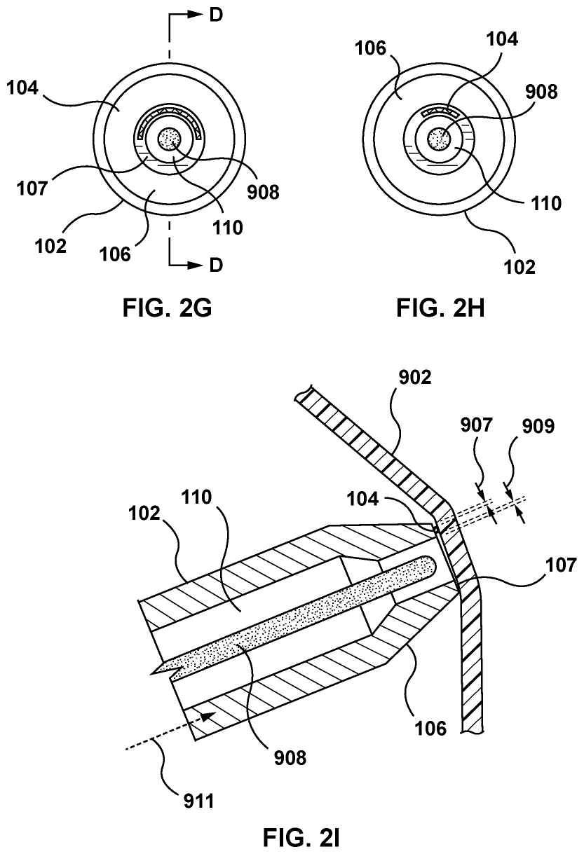

Referring to the embodiments as depicted in FIG. 1C, the elongated medical guidewire assembly 908 and the cautery device 104 are positioned relative to each other to avoid unwanted electrical short circuiting therebetween. The cautery device 104 is positioned at (fixedly positioned at) the distal end portion of the elongated medical dilator 102 proximate to the entrance of the elongated dilator cavity 110. Relative movement of the elongated medical guidewire assembly 908 should avoid inadvertent contact with the cautery device 104 (especially for the case where the cautery device 104 is activated). For improved safety, it may be recommended to retract the elongated medical guidewire assembly 908 into the elongated dilator cavity 110 and away from the entrance of the elongated dilator cavity 110 prior to activation of the cautery device 104. It will be appreciated that the geometry of the entrance of the elongated dilator cavity 110 may be adapted to avoid unwanted electrical contact between the elongated medical guidewire assembly 908 and the cautery device 104 (as depicted in the embodiments of FIG. 2F and FIG. 2I). For instance, the cautery device 104 may be set back (spaced apart) from the entrance of the elongated dilator cavity 110 (to avoid unwanted electrical contact between the cautery device 104 and the elongated medical guidewire assembly 908 once the elongated medical guidewire assembly 908 passes by the entrance of the elongated dilator cavity 110).

Referring to the embodiments as depicted in FIG. 1C, an electrical conductor 111 extends along a length of the elongated medical dilator 102. The electrical conductor 111 is electrically connected to the cautery device 104. The electrical conductor 111 is embedded along the dilator length of the elongated medical dilator 102. The elongated medical dilator 102 is configured to electrically insulate the electrical conductor 111 from the elongated medical guidewire assembly 908. The material of the body of the elongated medical dilator 102 may include a material having electrical insulation properties suitable for wiring, cabling and/or electrical shielding duties with sufficient safety performance properties (dielectric strength, thermal performance, insulation and corrosion, water and heat resistance) for safe performance to comply with medical, industrial and regulatory safety standards. Reference may be made to the following publication for consideration in the selection of a suitable material for the elongated medical dilator 102: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew. Sufficient electrical insulation may be required to mitigate environmental interference and/or operating interference from other elements. International standards for electrosurgical devices mandate the minimum electrical insulation performance of devices to protect both the patient and end user. For instance, to deliver about 270 Vrms to about 400 Vrms, it may be required to provide an insulation equivalent of approximately 0.00275 inch thick PTFE or more to satisfy current leakage requirements.

Referring to the embodiments as depicted in FIG. 1C, the cautery device 104 is in electrical communication with the proximal end of the electrical conductor 111 (wire). It may be preferred in some instances to use miniaturized electrical wires (for instance, from about 34 AWG to about 44 AWG). The material may include copper, stainless steel, nitinol, etc. A flat ribbon wire with a rectangular cross section may be utilized to minimize impact on the overall outer diameter of the elongated medical dilator 102. Total end-to-end DC resistance may be minimized. It may be preferred, for performance, to have a resistance under about 20 ohms. Depending on the embodiment of the elongated medical dilator 102, the elongated medical dilator 102 may feature metallic structures along its longitudinal length that are electrically conductive. The metallic elements may also be used as electrical connectors to simplify assembly.

Referring to the embodiments as depicted in FIG. 1C, the elongated medical dilator 102 may include a proximal connector (not depicted but known) configured to facilitate electrical connection of the back end of the elongated medical dilator 102 to an energy source (such as a radio frequency source or to a recorder device) and to the cautery device 104. A connector may be added axially over the back end of an exposed wire from the hub of the elongated medical dilator 102 (for example, using a speaker connector or alligator clip). Alternatively, a connector of a connector assembly may be clipped onto an electrical contact along the shaft of the elongated medical dilator 102 from the side (like a hairclip). Alternatively, an electrical connection may be provided by a close-fitting ancillary device (for example, the energy source may be connected to a supporting sheath, and there may be a matched internal connection made between the medical sheath 200 and the elongated medical dilator 102 when correctly positioned).

FIG. 2A, FIG. 2B and FIG. 2C depict front views (FIG. 2A and FIG. 2B) of embodiments of the elongated medical dilator 102 of FIG. 1A, and a side cross-sectional view (FIG. 2C) taken along a cross-sectional line B-B of FIG. 2A.

Referring to the embodiments as depicted in FIG. 2A, the elongated medical dilator 102 includes a leading face portion 107 (or a leading edge) positioned at the entrance of the elongated dilator cavity 110. The cautery device 104 is positioned on (over) a portion of the leading face portion 107. A portion of the cautery device 104 contacts a portion of the leading face portion 107. For instance, the cautery device 104 forms (includes) a semicircular shaped body that is positioned on a portion of the leading face portion 107. The cautery device 104 extends, preferably, between the opposite sides of the entrance leading into the elongated dilator cavity 110 of the elongated medical dilator 102; the cautery device 104 also extends (at least in part) along the dilation device 106 (along the outer surface of the dilation device 106).

Referring to the embodiments as depicted in FIG. 2B, the cautery device 104 forms (includes) a tab structure extending along a side portion of the dilation device 106 (on the outer surface of the dilation device 106). The cautery device 104 is positioned on a portion of the leading face portion 107.

Referring to the embodiments as depicted in FIG. 2C, the elongated medical dilator 102 includes a leading face portion 107 positioned at the entrance of the elongated dilator cavity 110. The cautery device 104 is positioned on a part of the leading face portion 107. The elongated medical dilator 102 is moved (pushed), by the user (doctor), toward the side wall of the tissue portion 902 of the living body 904 (of the patient) so that at least a part of the leading face portion 107 may contact the side wall of the tissue portion 902, and (at least a part of) the cautery device 104 contacts the side wall of the tissue portion 902. Once contact is made, the cautery device 104 may be activated to cauterize a part of the side wall of the tissue portion 902. It may be beneficial to temporarily withdraw (retract) the elongated medical guidewire assembly 908 away from the leading face portion 107 of the elongated medical dilator 102 before the cautery device 104 is activated.

FIG. 2D, FIG. 2E and FIG. 2F depict front views (FIG. 2D and FIG. 2E) of embodiments of the elongated medical dilator 102 of FIG. 1A, and a side cross-sectional view (FIG. 2F) taken along a cross-sectional line C-C of FIG. 2D.

Referring to the embodiments as depicted in FIG. 2D, the elongated medical dilator 102 includes a leading face portion 107 positioned at the entrance of the elongated dilator cavity 110. In an embodiment, the cautery device 104 is set back from the leading face portion 107. The cautery device 104 does not contact the leading face portion 107. For instance, the cautery device 104 forms (includes) a semicircular shaped body. The cautery device 104 extends, preferably, between the opposite sides of the entrance leading into the elongated dilator cavity 110 of the elongated medical dilator 102 along the dilation device 106 (along the outer surface of the dilation device 106).

Referring to the embodiments as depicted in FIG. 2E, the cautery device 104 forms (includes) a tab structure extending along a side portion of the dilation device 106 (on the outer surface of the dilation device 106). Preferably, the cautery device 104 is set back from the leading face portion 107. The cautery device 104 does not contact the leading face portion 107.

Referring to the embodiments as depicted in FIG. 2F, the elongated medical dilator 102 includes a leading face portion 107 positioned at the entrance of the elongated dilator cavity 110. Preferably, the cautery device 104 is set back from (and preferably does not contact) the leading face portion 107 in such a way that a safety gap 905 is formed between a leading edge of the cautery device 104 and a leading edge positioned proximate to the inner surface of the entrance of the elongated dilator cavity 110. The safety gap 905 assists in avoidance of an unwanted electrical short circuit between the elongated medical guidewire assembly 908 and the cautery device 104 (once the elongated medical guidewire assembly 908 is relatively moved along the movement direction 911, and once the cautery device 104 is activated, etc.). The safety gap 905 is configured to avoid an electrical short circuit between the elongated medical guidewire assembly 908 and the cautery device 104. The elongated medical dilator 102 is moved (pushed), by the user (doctor), toward the side wall of the tissue portion 902 of the living body 904 (of the patient) so that at least a part of the leading face portion 107 may contact the side wall of the tissue portion 902, and (at least a part of) the cautery device 104 contacts the side wall of the tissue portion 902. Once contact is made, the cautery device 104 may be activated to cauterize a part of the side wall of the tissue portion 902.

Referring to the embodiments as depicted in FIG. 2F, it may be beneficial to temporarily withdraw (retract) the elongated medical guidewire assembly 908 away from the leading face portion 107 of the elongated medical dilator 102 before the cautery device 104 is activated; however, the safety gap 905 may permit an option to not temporarily withdraw the elongated medical guidewire assembly 908 while the cautery device 104 is activated (if desired). For instance, for the case where the elongated medical guidewire assembly 908 is flexible, the elongated medical guidewire assembly 908 might inadvertently contact (and electrically short) the cautery device 104 if the elongated medical guidewire assembly 908 is extended exteriorly of the elongated medical dilator 102 and if the cautery device 104 is activated.

FIG. 2G, FIG. 2H and FIG. 2I depict front views (FIG. 2G and FIG. 2H) of embodiments of the elongated medical dilator 102 of FIG. 1A, and a side cross-sectional view (FIG. 2I) taken along a cross-sectional line D-D of FIG. 2G.

Referring to the embodiments as depicted in FIG. 2G, the elongated medical dilator 102 includes a leading face portion 107 positioned at the entrance of the elongated dilator cavity 110. The cautery device 104 is positioned on the leading face portion 107, and does not extend beyond the outer boundary of the leading face portion 107. For instance, the cautery device 104 forms (includes) a semicircular shaped body. The cautery device 104 extends, preferably, between the opposite sides of the entrance leading into the elongated dilator cavity 110 of the elongated medical dilator 102 along the dilation device 106 (along the outer surface of the dilation device 106).

Referring to the embodiments as depicted in FIG. 2H, the cautery device 104 forms (includes) a tab structure extending along a side portion of the dilation device 106 (on the outer surface of the dilation device 106). The cautery device 104 is positioned on the leading face portion 107, and does not extend beyond the outer boundary of the leading face portion 107.

Referring to the embodiments as depicted in FIG. 2I, the elongated medical dilator 102 includes a leading face portion 107 positioned at the entrance of the elongated dilator cavity 110. The cautery device 104 is set back from (and preferably does not contact) the inner and outer edges of the leading face portion 107 in such a way that an outer gap 907 and an inner gap 909 are formed between the opposite sides of the cautery device 104. The outer gap 907 and the inner gap 909 assist in avoidance of an unwanted electrical short circuit between the elongated medical guidewire assembly 908 and the cautery device 104 (once the elongated medical guidewire assembly 908 is moved (relatively) along the movement direction 911, and once the cautery device 104 is activated, etc.). The elongated medical dilator 102 is moved (pushed), by the user (doctor), toward the side wall of the tissue portion 902 of the living body 904 (of the patient) so that at least a part of the leading face portion 107 may contact the side wall of the tissue portion 902, and (at least a part of) the cautery device 104 contacts the side wall of the tissue portion 902. Once contact is made, the cautery device 104 may be activated to cauterize a part (portion) of the side wall of the tissue portion 902.

Referring to the embodiments as depicted in FIG. 2I, it may be beneficial to temporarily withdraw (retract) the elongated medical guidewire assembly 908 away from the leading face portion 107 of the elongated medical dilator 102 before the cautery device 104 is activated; however, the outer gap 907 and the inner gap 909 may permit an option not to temporarily withdraw the elongated medical guidewire assembly 908 while the cautery device 104 is activated (if desired). For instance, for the case where the elongated medical guidewire assembly 908 is flexible, the elongated medical guidewire assembly 908 might inadvertently contact (and electrically short) the cautery device 104 if the elongated medical guidewire assembly 908 is extended exteriorly of the elongated medical dilator 102 and if the cautery device 104 is activated.

Referring to the embodiments as depicted from FIG. 2A to FIG. 2I, for the case where the cautery device 104 includes a ring-shaped electrode positioned at the distal end of the elongated medical dilator 102, there may be a risk of tissue coring (that is, the unwanted cutting of a tissue plug or tissue particle), which may result in the release of embolic material into the bloodstream. As such, it may be desirable for the cautery device 104 to not circumscribe an area or volume of tissue (thereby avoiding the formation of the tissue particle). For instance, having the cautery device 104 include a semicircular shaped electrode or a tab-shaped structure (electrode arrangement) may mitigate tissue coring while delivering sufficient current (thermal energy) to the adjacently located tissue to puncture a pilot hole (tissue passageway) for subsequent tissue dilation (by the dilation device 106). Examples of the above illustrate variations of the cautery device 104 that are open to internal environments, and those which are insulated from one or more of the faces with a sufficient gap of insulating material.

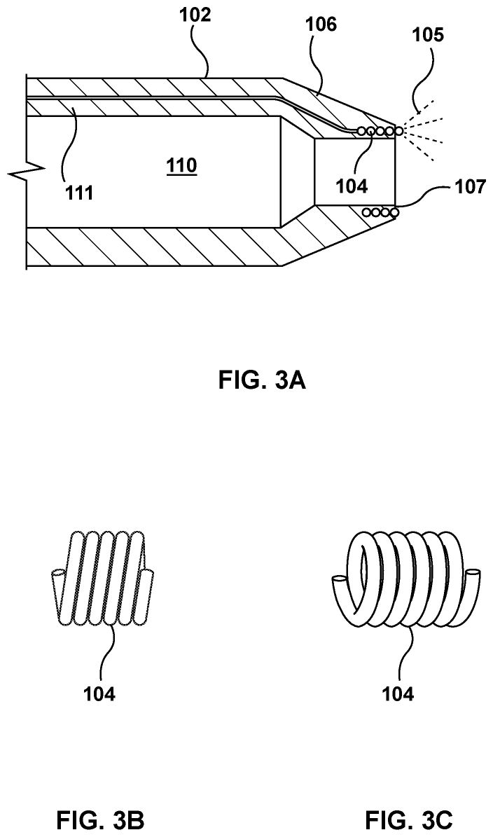

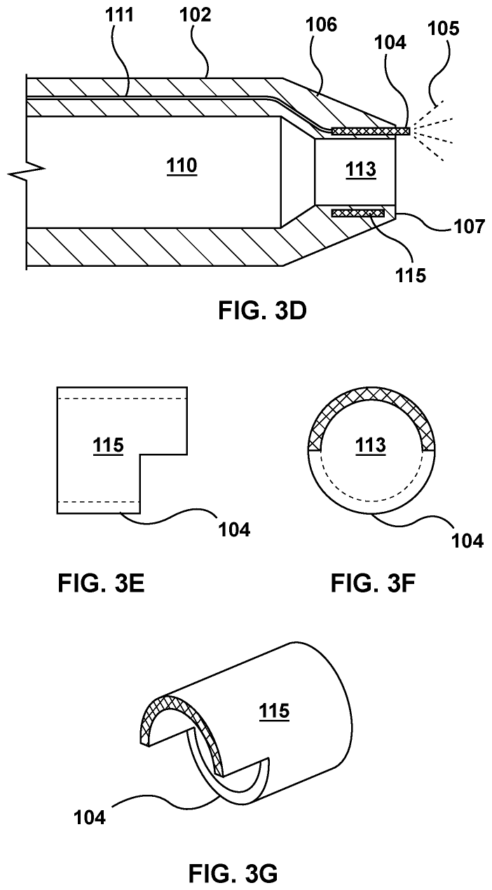

FIG. 3A, FIG. 3B and FIG. 3C depict a cross-sectional view (FIG. 3A) of an embodiment of the elongated medical dilator 102 of FIG. 1, a side view (FIG. 3B) and a perspective view (FIG. 3C) of the embodiments of the elongated medical dilator 102 of FIG. 3A.

Referring to the embodiments as depicted in FIG. 3A to FIG. 3C, the cautery device 104 is embedded (at least in part) in the distal tip portion (leading edge) of the dilation device 106, and the dilation device 106 is positioned at the distal tip portion of the elongated medical dilator 102. Generally, the cautery device 104 is embedded in the distal tip portion (leading edge) of the elongated medical dilator 102, and the dilation device 106 extends from the distal tip portion of the elongated medical dilator 102. At least a portion of the cautery device 104 is exposed for contact with tissue of the patient. At least a portion of the cautery device 104 is exposed and extends forwardly from the distal tip portion of the elongated medical dilator 102 (or extends forwardly from the distal tip portion of the dilation device 106). For instance, the portion of the cautery device 104 that is exposed provides an exposed component (for contact with tissue) that avoids unwanted tissue coring (avoids the formation of a stray tissue portion that might travel in the bloodstream). The cautery device 104 is configured to (shaped to) not form a tissue core (tissue coring) from the tissue (to be cauterized) once the cautery device 104 is activated. It will be appreciated that it may be desirable to avoid embolic stroke. An embolic stroke refers to the blockage of an artery by an embolus, a traveling particle or debris in the arterial bloodstream originating from elsewhere.

Referring to the embodiments as depicted in FIG. 3A to FIG. 3C, the cautery device 104 is overmolded by the elongated medical dilator 102. Overmolding is a process where a single part is created using two or more different materials in combination, in which the first material (sometimes referred to as the substrate) is partially or fully covered by subsequent materials (an overmold material) during the manufacturing process. Overmolding is effectively the use of layering effects in polymer application techniques. This process utilizes a liquidous resin to add one or more additional layers of shape and structure to an existing component. The resin may include a polymer that has been heated to a temperature just above its glass transition temperature.

Referring to the embodiments as depicted in FIG. 3A to FIG. 3C, the electrical conductor 111 is embedded in the material of body of the elongated medical dilator 102, and is electrically connected to the cautery device 104. The material of the body of the elongated medical dilator 102 is electrically insulated (so that the electrical conductor 111 is not electrically shorted).

Referring to the embodiment as depicted in FIG. 3A, the cautery device 104 is actuated (via application of electrical energy via the electrical conductor 111) so that thermal energy 105 is emitted from the exposed portion of the cautery device 104.

Referring to the embodiments as depicted in FIG. 3B and FIG. 3C, the cautery device 104 includes an uninsulated electrical coil (a coiled electrical wire having no layer of electrical insulation material).

FIG. 3D, FIG. 3E, FIG. 3F and FIG. 3G depict a cross-sectional view (FIG. 3D) of an embodiment of the elongated medical dilator 102 of FIG. 1, a side view (FIG. 3E), a front view (FIG. 3F) and a side perspective view (FIG. 3G) of the embodiments of the elongated medical dilator 102 of FIG. 3D.

Referring to the embodiments as depicted in FIG. 3D to FIG. 3G, the cautery device 104 includes a cautery device body 115 defining a cautery device hole 113 extending through the central portion of the device body 115. A substantial portion of the cautery device 104 is embedded in the frontal tip portion (distal end portion) of the elongated medical dilator 102. The device hole 113 is coaxially aligned with the elongated dilator cavity 110 of the elongated medical dilator 102. The device body 115 defines (forms) a frontal extended section (a sloped section or tapered forward sloped portion). A portion of the frontal sloped section (tapered forward sloped) of the device body 115 is exposed (for tissue contact) and extends forwardly from the distal end portion of the elongated medical dilator 102.

Referring to the embodiments as depicted in FIG. 3E, FIG. 3F and FIG. 3G, the cautery device 104 includes the device body 115 shaped as a tubular element having a frontal extended section (to be exposed for cauterization of the tissue).

Referring to the embodiments as depicted from FIG. 3A to FIG. 3G, the cautery device 104 (such as a platinum coil) may be embedded (at least in part) into the distal end of the elongated medical dilator 102. The cautery device 104 is configured for non-coring puncture of tissue. The cautery device 104 may include a metallic coil and/or a section of an open-ended tube with an asymmetric face embedded (at least in part) into the distal end of the elongated medical dilator 102. The leading edge of the cautery device 104 may present an exposed portion of a conductive element, or the insulation of the elongated medical dilator 102 material may be partially removed to expose a cutting surface of the cautery device 104. The cautery device 104 may provide an exposed conductive element extending from the proximal end of the elongated medical dilator 102.

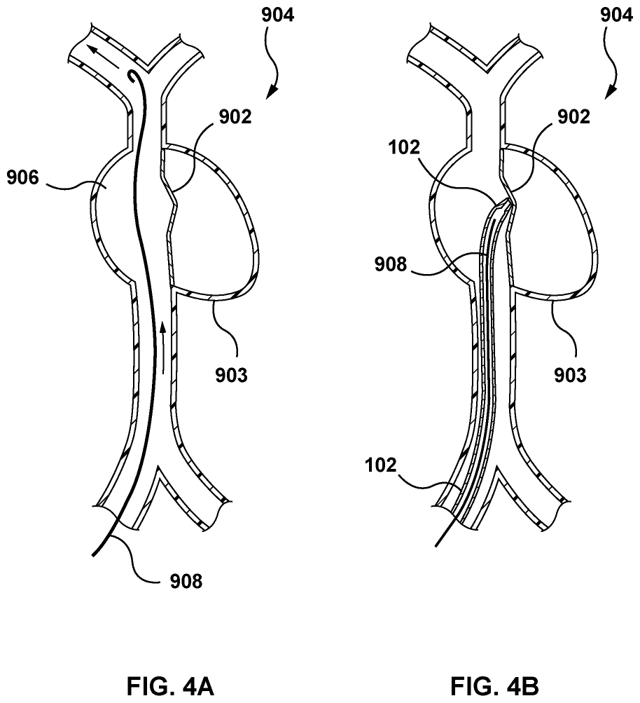

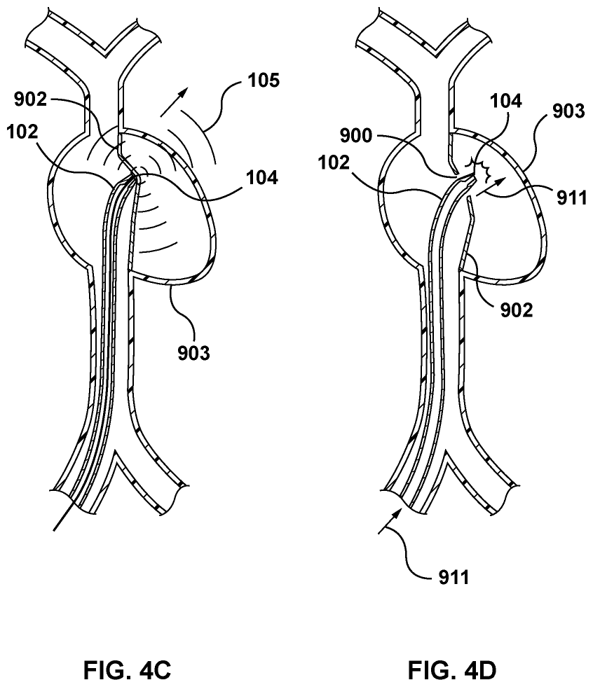

FIG. 4A, FIG. 4B, FIG. 4C and FIG. 4D depict a workflow using the embodiments of the elongated medical dilator of FIG. 1A.

Referring to the embodiment as depicted in FIG. 4A, the elongated medical guidewire assembly 908 is inserted into the confined space 906 defined by the living body 904 (of the patient), such as a vein of the patient. The elongated medical guidewire assembly 908 is moved (translated) toward the tissue portion 902 of the heart 903 and positioned proximate to the tissue portion 902 of the living body 904.

Referring to the embodiment as depicted in FIG. 4B, once the elongated medical guidewire assembly 908 is kept stationary, the elongated dilator cavity 110 of the elongated medical dilator 102 is positioned (installed) to receive the elongated medical guidewire assembly 908. The elongated medical dilator 102 is moved along (over) the elongated medical guidewire assembly 908 toward the tissue portion 902 positioned in the heart 903. The cautery device 104 is not activated during the movement of the elongated medical dilator 102 toward the tissue portion 902 located in the heart 903.

Referring to the embodiment as depicted in FIG. 4C, movement of the elongated medical dilator 102 results in positioning of the cautery device 104 in an abutment relationship with the tissue portion 902. Once positioned, the cautery device 104 is activated and the cautery device 104 emits the thermal energy (or RF energy) 105 toward the tissue portion 902.

Referring to the embodiment as depicted in FIG. 4D, activation of the cautery device 104 results in formation of the tissue passage 900 (specifically, an initial relatively small pilot hole) in the tissue portion 902. Further movement of the elongated medical dilator 102 (along the movement direction 911) forward into the tissue passage 900 results in further widening (reaming) of the tissue passage 900. It will be appreciated that the movement of the elongated medical guidewire assembly 908 and/or the elongated medical dilator 102 may be tracked using conventional techniques for medical imaging, such as echo cardiology, fluoroscopy, etc., and any equivalent thereof.

Referring to the embodiments as depicted from FIG. 4A to FIG. 4D, there is depicted a workflow using the elongated medical dilator 102. After the elongated medical guidewire assembly 908 (a standard non-puncture guidewire) is tracked from the femoral veins to the heart 903, the elongated medical dilator 102 (along with an ancillary sheath, if so desired) is/are advanced to the cardiac silhouette. Contact between the elongated medical dilator 102 and the target tissue is confirmed before delivering thermal energy (such as radio frequency energy) to puncture the tissue. The elongated medical dilator 102 and the elongated medical guidewire assembly 908 (along with the medical sheath 200, if utilized) are all advanced to the left heart.

Referring to the embodiments as depicted from FIG. 4A to FIG. 4D, there is depicted a method including and not limited to (comprising) an operation of utilizing a cautery device 104 of an elongated medical dilator 102 for selectively forming, by cauterization, a tissue passage 900 through a tissue portion 902 of a living body 904. The method also includes an operation of utilizing a dilation device 106 of the elongated medical dilator 102, in which the dilation device 106 is positioned relative to the cautery device 104, for dilating the tissue passage 900 once the cautery device 104 selectively forms the tissue passage 900.

Referring to the embodiments as depicted from FIG. 4A to FIG. 4D, there is depicted a method including and not limited to (comprising) an operation of utilizing a cautery device 104 of an elongated medical dilator 102 for selectively forming, by cauterization, a tissue passage 900 through a tissue portion 902 of a living body 904 once the elongated medical dilator 102 is received into a confined space 906 defined by the living body 904, and the cautery device 104 is positioned proximate to the tissue portion 902. The method also includes an operation of utilizing a dilation device 106 of the elongated medical dilator 102, in which the dilation device 106 is positioned relative to the cautery device 104, for dilating the tissue passage 900 once the cautery device 104 selectively forms the tissue passage 900, and the dilation device 106 is urged to move toward, and to make intimate contact with, the tissue passage 900 formed by the cautery device 104.

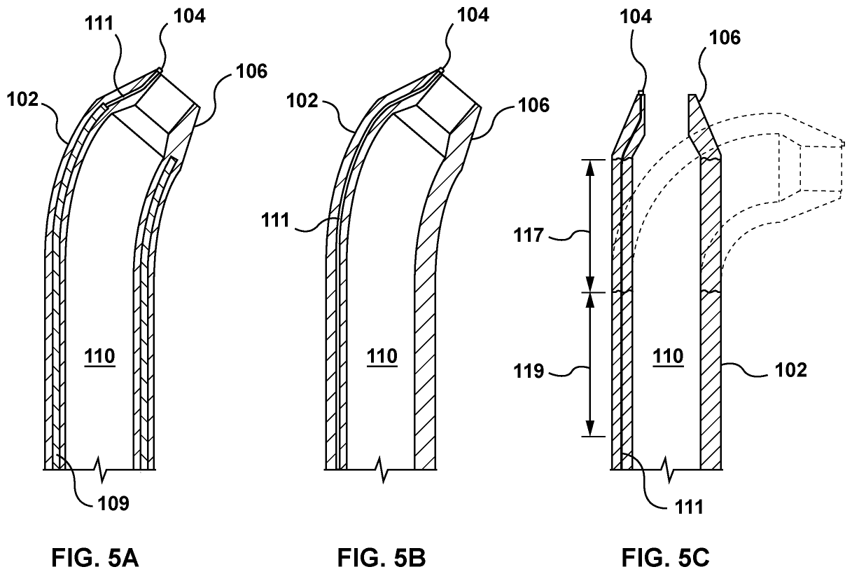

FIG. 5A, FIG. 5B and FIG. 5C depict cross-sectional views of the embodiments of the elongated medical dilator 102 of FIG. 1A.

Referring to the embodiments as depicted from FIG. 5A to FIG. 5C, there are depicted three (3) different embodiments of the elongated medical dilator 102, in which FIG. 5A depicts a standard embodiment, FIG. 5B depicts a reinforced embodiment and FIG. 5C depicts a flexible embodiment.

Referring to the embodiment as depicted in FIG. 5A, the elongated medical dilator 102 includes a shapeable electrically-conductive material 109 (hereafter referred to as the material 109) embedded in the body of the elongated medical dilator 102. It will be appreciated that for the case where the material is too hard or brittle, reshaping may not be permitted, and there may be a risk of losing electrical contact; however, there exists a balance of materials that may be sufficiently stiff, and yet still shapable and spring like (these materials are known to persons of skill in the art). In this manner, the elongated medical dilator 102 may be shaped by the user (into a desired shape) before insertion of the elongated medical dilator 102 into the body of the patient. The material 109 extends along a longitudinal length of the elongated medical dilator 102. A distal portion of the material 109 is electrically connected to the cautery device 104 (such as via the electrical conductor 111). The material 109 is configured to permit flexed side-to-side movement of the elongated medical dilator 102. The elongated medical dilator 102 may include a uniform polymer such as HDPE (high-density polyethylene) or polyethylene high-density (PEHD), which is a thermoplastic polymer produced from the monomer ethylene. It is sometimes called alkathene or polythene. HDPE is corrosion-resistant. The elongated medical dilator 102 may include a uniform polymer with relatively thick walls with an outer diameter of about 0.111 inches, and an inner diameter of about 0.056 inches, or having an 8.5 Fr (French catheter scale) sizing. Referring to the embodiment as depicted in FIG. 5A, the elongated medical dilator 102 is configured for manual shapability and/or for electrical conductivity. For one embodiment, an electrically conductive shapable element (such as, a stainless steel or a plastically deformable material) is embedded (preferably entirely or at least in part) in the walls of the elongated medical dilator 102. However, it will be appreciated that, in accordance with another embodiment, a relatively stiffer and relatively shapable element is separated from an electrical connector (also called a proximal connector, known and not depicted) of the elongated medical dilator 102. It will be appreciated that there are many other arrangements (not depicted) as alternatives for the embodiment as depicted in FIG. 5A (for which the person of skill in the art would be able to derive given the description for the embodiment depicted in FIG. 5A).

Referring to the embodiment as depicted in FIG. 5B, the elongated medical dilator 102 includes the electrical conductor 111 extending along a longitudinal length of the elongated medical dilator 102 (and is embedded within the body of the elongated medical dilator 102). The body of the elongated medical dilator 102 includes an electrically insulated material. The elongated medical dilator 102 may include a stainless steel hypotube positioned within the body of the elongated medical dilator 102; in this arrangement (manner), the elongated medical dilator 102 is configured to provide shapeability and may additionally function as an electrical conductor. A hypotube is a long metal tube with micro-engineered features along its length.

Referring to the embodiment as depicted in FIG. 5C, the elongated medical dilator 102 is configured to flex while the elongated medical dilator 102 is positioned within the body of the patient. The elongated medical dilator 102 includes a relatively flexible section 117 positioned at a distal portion (front portion) of the elongated medical dilator 102. The relatively flexible section 117 provides relatively lower stiffness. The elongated medical dilator 102 also includes a relatively stiffer section 119 positioned adjacent to the relatively flexible section 117. The relatively stiffer section 119 provides relatively higher stiffness (that is, relative to the relatively flexible section 117). The relatively stiffer section 119 is spaced apart from the distal portion (front portion) of the elongated medical dilator 102. Once the elongated medical dilator 102 is flexed while inside the body of the patient, the cautery device 104 and the dilation device 106 are repositioned to the desired target region.

Referring to the embodiment of FIG. 5C, the wall materials of the elongated medical dilator 102 may be configured to provide flexibility, and may be preferentially stiffer or softer along a longitudinal length of the elongated medical dilator 102; this arrangement may be useful when paired with (used with) a steerable sheath (known and not depicted) that may deflect a soft distal end of the elongated medical dilator 102 towards the tissue of interest. The soft section may be fabricated by a section of LDPE bonded end to end with HDPE (high-density polyethylene). Low-density polyethylene (LDPE) is a thermoplastic made from the monomer ethylene. The elongated medical dilator 102 may include many types of components, such as a flexible neck, a hypotybe reinforcement, etc., and any equivalent thereof. It will be appreciated that there are many suitable materials usable in the fabrication of the components of the elongated medical dilator 102, such as polyethylenes, etc., and any equivalent thereof.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open-ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus comprising:
an elongated medical dilator including:
a cautery device being configured to selectively form, by cauterization, a tissue passage through a tissue portion of a living body; and
a dilation device being positioned relative to the cautery device, and the dilation device being configured to dilate the tissue passage once the cautery device selectively formed the tissue passage.

2. An apparatus comprising:
an elongated medical dilator including:
a cautery device being configured to selectively form, by cauterization, a tissue passage through a tissue portion of a living body once the elongated medical dilator is received into a confined space defined by the living body, and the cautery device is positioned proximate to the tissue portion; and
a dilation device being positioned relative to the cautery device, and the dilation device being configured to dilate the tissue passage once the cautery device selectively formed the tissue passage, and the dilation device is urged to move toward, and to make intimate contact with, the tissue passage formed by the cautery device.

3. The apparatus of claim 2, wherein:
the elongated medical dilator defines an elongated dilator cavity extending along a length of the elongated medical dilator; and
the elongated dilator cavity is configured to receive an elongated medical guidewire assembly therein, in which the elongated medical guidewire assembly is configured to be inserted into the confined space defined by the living body.

4. The apparatus of claim 3, wherein:
the elongated medical guidewire assembly and the cautery device avoid unwanted electrical short circuiting therebetween.

5. The apparatus of claim 3, wherein:
a geometry of an entrance of an elongated dilator cavity is adapted to avoid unwanted electrical contact between the elongated medical guidewire assembly and the cautery device.

6. The apparatus of claim 3, wherein:
the elongated medical dilator includes a leading face portion positioned at an entrance of the elongated dilator cavity; and
the cautery device is positioned on a portion of the leading face portion.

7. The apparatus of claim 1, wherein:
the elongated medical dilator includes a distal tip having a leading face portion; and
the cautery device is fixedly positioned at the leading face portion of the distal tip of the elongated medical dilator.

8. The apparatus of claim 1, wherein:
the cautery device includes an electrode.

9. The apparatus of claim 1, further comprising:
an elongated electrical conductor being aligned along a length of the elongated medical dilator.

10. The apparatus of claim 1, further comprising:
an elongated electrical conductor being aligned along a length of a body portion of the elongated medical dilator.

11. The apparatus of claim 1, wherein:
the elongated medical dilator includes a proximal terminal; and
the cautery device is configured to receive energy from the proximal terminal via an elongated electrical conductor.

12. The apparatus of claim 1, wherein:
the cautery device is configured to avoid formation of a tissue core from the tissue portion to be cauterized once the cautery device is activated.

13. The apparatus of claim 1, wherein:
a medical sheath defines an elongated interior passageway configured to receive the elongated medical dilator.

14. The apparatus of claim 1, wherein:
the dilation device is configured to mechanically ream a passageway through tissue.

15. The apparatus of claim 1, wherein:
the dilation device includes a sloped surface extending from a distal portion of the elongated medical dilator.

16. The apparatus of claim 15, wherein:
the cautery device includes a semicircular shaped body that is positioned on a portion of a leading face portion of the elongated medical dilator.

17. The apparatus of claim 15, wherein:
the cautery device extends between opposite sides of an entrance leading into an elongated dilator cavity of the elongated medical dilator, and extends, at least in part, along an outer surface of the dilation device.

18. The apparatus of claim 1, wherein:
the cautery device includes a tab structure extending, at least in part, along a side portion of the dilation device and extends, at least in part, on an outer surface of the dilation device.

19. The apparatus of claim 16, wherein:
the cautery device does not contact the leading face portion of the elongated medical dilator.

20. The apparatus of claim 1, wherein:
the cautery device includes a semicircular shaped body.

21. The apparatus of claim 1, wherein:
the cautery device includes a tab structure extending along a side portion of the dilation device on an outer surface of the dilation device.

22. The apparatus of claim 1, wherein:
the elongated medical dilator includes a leading face portion positioned at an entrance of an elongated dilator cavity; and
the cautery device is set back from, and does not contact, the leading face portion in such a way that a safety gap is formed between a leading edge of the cautery device and a leading edge positioned proximate to an inner surface of an entrance of the elongated dilator cavity; and
the safety gap is configured to avoid an electrical short circuit between an elongated medical guidewire assembly and the cautery device, in which an elongated dilator cavity is configured to receive the elongated medical guidewire assembly.

23. The apparatus of claim 1, wherein:
the cautery device is embedded, at least in part, in a distal tip portion of the dilation device; and
the dilation device is positioned at the distal tip portion of the elongated medical dilator.

24. The apparatus of claim 1, wherein:
the elongated medical dilator includes an electrically-conductive material having a distal portion being electrically connected to the cautery device.

25. The apparatus of claim 1, wherein:
a medical sheath defines an elongated interior passageway configured to receive the elongated medical dilator; and
a geometry of an entrance of the medical sheath is adapted to avoid unwanted electrical contact between the elongated medical guidewire assembly and the cautery device.

26. An apparatus comprising:
an elongated medical dilator having a dilator length being configured to be received into a confined space defined by a living body; and
the elongated medical dilator including a cautery device being configured to be positioned proximate to a tissue portion of the living body once the elongated medical dilator is received into the confined space defined by the living body; and
the cautery device also being configured to selectively cauterize the tissue portion of the living body in such a way that the cautery device, once actuated, forms, by cauterization, a tissue passage through the tissue portion once the cautery device is positioned proximate to the tissue passage and is selectively actuated; and
the elongated medical dilator also including a dilation device being positioned relative to the cautery device, and being configured to be received into the confined space defined by the living body; and
the dilation device also being configured to support the cautery device; and
the dilation device being configured to dilate the tissue passage once the cautery device selectively formed the tissue passage, and the dilation device is urged to move toward, and to make intimate contact with, the tissue passage formed by the cautery device.

27. A method, comprising:
utilizing a cautery device of an elongated medical dilator, and the cautery device configured to selectively form, by cauterization, a tissue passage through a tissue portion of a living body; and
utilizing a dilation device of the elongated medical dilator, in which the dilation device is positioned relative to the cautery device, and the dilation device configured to dilate the tissue passage once the cautery device selectively formed the tissue passage.

28. A method, comprising:
utilizing a cautery device of an elongated medical dilator for selectively forming, by cauterization, a tissue passage through a tissue portion of a living body once the elongated medical dilator is received into a confined space defined by the living body, and the cautery device is positioned proximate to the tissue portion; and
utilizing a dilation device of the elongated medical dilator, in which the dilation device is positioned relative to the cautery device, for dilating the tissue passage once the cautery device selectively formed the tissue passage, and the dilation device is urged to move toward, and to make intimate contact with, the tissue passage formed by the cautery device.
